(19) United States
(12) Patent Application Publication
(10) Pub. No.: US 2026/0029383 A1
(43) Pub. Date: Jan. 29, 2026

(54) Air-coupled ultrasonic metrology platform for in-line characterization of battery porous electrodes

(71) Applicant: Drexel University, Philadelphia, PA (US)

(72) Inventors: Wesley Kuo-Wei Chang, Philadelphia, PA (US); André Matthew Ricarte Tayamen, Philadelphia, PA (US); Samuel Amsterdam, Philadelphia, PA (US)

(73) Assignee: Drexel University, Philadelphia, PA (US)

(21) Appl. No.: 19/282,456

(22) Filed: Jul. 28, 2025

Related U.S. Application Data
(60) Provisional application No. 63/676,578, filed on Jul. 29, 2024.

(51) Int. Cl. G01N 29/44; G01N 29/024; G01N 29/09; G01N 29/265; G01N 33/2045

(52) U.S. Cl. CPC G01N 29/4463; G01N 29/024; G01N 29/09; G01N 29/265; G01N 33/2045; G01N 2291/0234; G01N 2291/0289

(57) ABSTRACT

An air-coupled ultrasonic scanning platform for an air-coupled ultrasonic non-contact metrology of battery electrodes that maps the local variation in density and thickness, and detect defects during electrode casting and drying, includes: piezocomposite air-coupled transducers (Ultran) at 0.5 to 1 MHz that provide sufficient air coupling to enable sound waves to travel through air from a transmitting transducer, through the thin film electrode coated on metal current collector, and then into the receiving transducer. Non-contact, air-coupled ultrasound may be used as a technique for evaluating battery electrode films. An analytical model was derived from fundamental acoustic wave propagation principles to determine acoustic density. Voltage gain or acoustic density maps of electrode films revealed features that are not visually apparent, attributed to mass gradients.

### BACKGROUND

The growing demand for electric vehicles (EVs) has prompted a significant increase in battery production capacity, towards a projected 2.6 TWh of global battery production by 2030. With a current energy requirement of 10 GWh per GWh capacity at best, improved manufacturing process efficiency is also critical for reducing the total energy usage required. A techno-economic analysis of battery production asserts five factors affecting the roll-to-roll (R2R) capacity of a battery production plant (in decreasing impact): 1) R2R speed, 2) electrode thickness, 3) days of active operation, 4) cathode specific capacity, and 5) end-of-line scrap rate. Factors 1, 2, and 4 are specific processing parameters that can be modified, but factors 3 and 5 depend on the output of the production process. The end-of-line scrap rate refers to the amount of material that is unusable for end products, which can be due to trimming waste, process startup, defective or off-spec electrodes. In contrast, the number of days of operation deals with machine availability and is limited by maintenance requirements or offline testing of products. Implementing non-destructive evaluation (NDE) techniques in the fabrication process can minimize scrap by enabling the real-time detection of defects or defective materials, thereby maximizing factory days of active operation.

Several methods have been demonstrated for non-destructive evaluation of battery electrodes, such as laser-based thickness measurements, optical imaging, and infrared (IR) thermography for electrode defects, and radiation-based NDE and ultrasound for mass loading. Among these techniques, ultrasonic testing provides rich information in the form of signal amplitude, time, and frequency, which can be further processed to obtain data about electrode density, defects, and mechanical properties.

Ultrasound has been explored as a non-destructive method to evaluate fabricated batteries. Amplitude and time-of-flight measurements correlate to the state-of-charge (SOC) and state-of-health (SOH) of cells during charge and discharge. Variations in amplitude within pouch cells have been shown to correlate with wetting behavior. Lithium plating was detected using operando ultrasonic testing in lithium nickel manganese cobalt oxide/copper (NMC/Cu) pouch cells. These methods generally require a coupling gel or fluid to efficiently transfer sound waves through the source-medium-detector path, which limits their application to enclosed, fully fabricated batteries rather than individual components.

The scaling up of Li-ion and beyond-Li-ion battery manufacturing plants involves precise roll-to-roll manufacturing of thin (less than 100 microns) porous electrodes. Conventional battery electrodes are made by mixing slurries of active materials, conductive carbon, polymer (poly(vinylidene fluoride), or PVDF) binder, and organic solvent (N-methyl-2-pyrrolidone, or NMP), and casting slurries as thin films onto current collector substrates. The electrodes are dried and then used for battery cell assembly by winding and stacking.

Poor manufacturing could induce heterogeneities in electrode microstructural properties, such as variable active material ratios and electrode cracking. These variations can result in changes in the density distribution of the thin film electrodes. Further, slurry casting may easily absorb factory impurities and contaminants, such as metal shavings and current collector burrs during electrode slitting.

For these reasons, precise and accurate quality checking and quality assurance (QA/QC) is required, preferably in-line to detect manufacturing defects rapidly. Conventional manufacturing plants have optical cameras and laser thickness gauges, which are ineffective at finding buried defects (e.g. voids) or local density variations that do not present visual differences. X-rays and other spectroscopic imaging tools are high-resolution but require ex situ sampling and are time- and cost-intensive. Ultrasound has recently been introduced into battery diagnostics as a non-destructive probe of cell state-of-health (SOH) and state-of-charge (SOC), because sound transmission speeds are correlated to density and modulus (which change as a battery is charged and discharged). This has primarily been limited to using contact-mode transducers by placing piezoelectric transducers directly on the face of a closed-form battery (pouch cell), or within a fluid immersion bath. Standard ultrasonic transducers require a liquid or solid couplant to maintain high transmission energy from the transducers through the cells.

Several related patents and applications include the following, which are incorporated by reference as if fully set forth herein: U.S. Pat. No. 11,855,265; US Pub. No. 2023/0393097A1; US Pub. No. 2023/0258608A1; US Pub. No. 2023/0258603A1; US Pub. No. 2023/0221285A1; U.S. Pat. No. 11,549,919; and US Pub. No. 2022/0155262A1.

### SUMMARY OF THE EMBODIMENTS

The inventors have found capabilities of air-coupled ultrasound scanning for quantitative metrology of electrodes by starting from an analytical model of acoustic wave propagation from fundamental principles. The non-contact, non-destructive, and rapid acquisition rates make the technique potentially suitable for in-line electrode manufacturing quality assurance and quality checking.

A non-invasive and non-destructive ultrasonic method for producing/improving lithium metal batteries improves ultrasound for gauging the state-of-charge and state-of-health of Li-ion batteries. The technique uses high frequency (2.25 MHz, for instance) contact-mode transducers (the standard used in geophysics and biomedical fields). Unlike other research groups that have primarily focused on using standard contact-mode transducers, which provide good resolution but require a liquid couplant to effectively transmit waves through the battery, the current method uses air-coupled transducers, which are more compatible with direct in-line electrode metrology.

An air-coupled ultrasonic scanning platform for an air-coupled ultrasonic non-contact metrology of battery electrodes maps the local variation in density and thickness. It detects defects during electrode casting and drying, includes: piezocomposite air-coupled transducers (Ultran) at 0.5 to 1 MHz that provide sufficient air coupling to enable sound waves to travel through air from a transmitting transducer, through the thin film electrode coated on metal current collector, and then into the receiving transducer.

### BRIEF DESCRIPTION OF THE DRAWINGS

FIG. 1a shows a schematic of an acoustic wave propagation in transmission mode through a battery electrode in air.

FIG. 1b shows the path of acoustic wave propagation in the experimental setup.

FIG. 2 shows an example of a tone burst ultrasound transmission waveform collected at one position during a raster scan, as demonstrated by the grid, and the resulting acoustic voltage gain map and voltage gain distribution (probability density) after post-processing.

FIG. 3A shows the voltage gain map for a lab-made electrode.

FIG. 3B shows an optical image of the lab-made electrode.

FIG. 3C shows an acoustic image of a double-sided, machine-made NMC electrode.

FIG. 3D shows the voltage gain distribution comparison between the machine-made and the lab-made electrode, showing a clear difference in voltage gain distribution.

FIG. 3E shows the most homogeneous region of the lab-made electrode from FIG. 3A.

FIG. 3F shows the voltage gain distribution of the most homogeneous region of the lab-made electrode versus the machine-made electrode, where the lab-made electrode continues to show a more skewed distribution.

FIGS. 4A and 4B show the same electrode as cast and after calendaring, with a 40 μm gap, with the calendering process possibly inducing a wrinkle in the electrode from uneven rollers.

FIG. 4C shows the corresponding probability density distribution, where the calendered electrodes show a clear shift in the voltage gain distribution.

FIGS. 5A-5D shows the acoustic images and associated optical images of aluminum shavings on top of the electrode (FIG. 5A, FIG. 5B) and embedded shavings (FIG. 5C, FIG. 5D).

FIG. 5E shows the voltage gain distribution.

FIG. 6A shows the acoustic density map of a calendered electrode using the model herein to convert voltage gain to acoustic density. FIG. 6B shows the corresponding acoustic density distribution of the electrode.

FIG. 7A shows an acoustic amplitude map of an electrode with the electrode facing the transmitter (“front” ultrasound wave goes through the electrode first).

FIG. 7B shows an acoustic amplitude map of an electrode with the A1 current collector facing the transmitter (“back” ultrasound wave goes through A1 current collector first).

FIG. 7C shows the corresponding amplitude distribution curves including a zoomed-in view.

FIG. 8 shows drawings of a transducer holder with a minimal transducer distance, preferably of 15 mm.

FIG. 9 shows an ultrasound signal from transmission through air with transducer distances of 15 mm (top) and 25 mm (bottom).

### DETAILED DESCRIPTION OF THE EMBODIMENTS

Ultrasound Model from Fundamental Principles

Sound waves in this ultrasound model are assumed to have normal incidence with the material and are measured in transmission mode. When modeling an acoustic pressure wave propagating through an interface between two materials, an essential boundary condition is that the materials remain in contact. As a result, acoustic pressure and particle velocity on both sides must be conserved (Equations 1 and 2). Three quantities may be considered at each interface: 1) the incident pressure, P_{incident}, which indicates the pressure just as the wave interacts with the interface, 2) the reflected pressure, P_{reflected}, which indicates the pressure of the wave propagating back on the side of the incident pressure, and 3) the transmitted pressure, P_{transmitted}, which indicates the pressure of the wave propagating through the interface.

$${P}_{incident} + {P}_{r e f l e c t e d} = {P}_{transmitted} \qquad ( EQ . 1 )$$

$${v}_{incident} + {v}_{reflected} = {v}_{t r a n s m i t t e d} \qquad ( EQ . 2 )$$

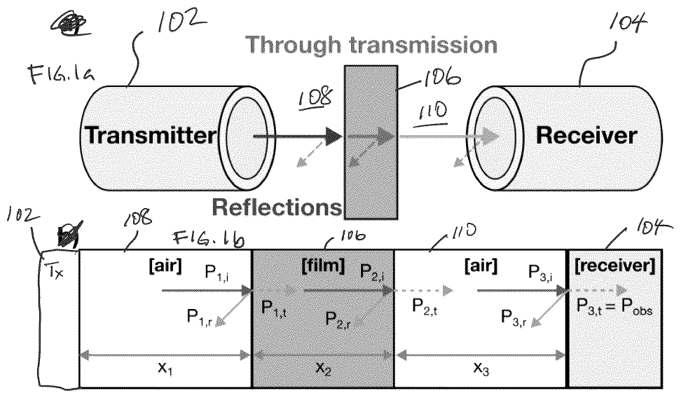

FIG. 1a shows a schematic of an acoustic wave propagation in transmission mode through a battery electrode in air, where a transmitter 102 transmits to a receiver 104 through a film 106, and air 108, 110 on either side of the film 106.

FIG. 1b is a related representation of acoustic qualities of model derivation, with arrows indicating a general direction of wave propagation. Reflected waves are normal to the interface but shown at an angle for clarity.

Since each pressure cannot easily be measured at each interface, the reflection coefficient, r, and transmission coefficient, t, are used to describe the reflection and transmission, respectively, of an acoustic wave across an interface. Furthermore, these coefficients are determined by acoustic impedances, Z, of the materials at the interface. This property relates the particle vibrations in a material to the applied pressure and is equivalent to the product of the material's mass density and speed of sound (Equation 3). The reflection and transmission coefficients can be easily expressed in terms of impedances by Equations 4 and 5.

$$Z = ρ c \qquad ( EQ . 3 )$$

$$r = Preflected / Pincident = ( {Z}_{2} - {Z}_{1} ) / ( {Z}_{2} + {Z}_{1} ) \qquad ( EQ . 4 )$$

$$t = Ptransmitted / Pincident = ( 2 {Z}_{2} / ( {Z}_{2} + {Z}_{1} ) \qquad ( EQ . 5 )$$

Z₁ indicates the acoustic impedance of the material where the wave initially propagates, and Z₂ indicates the acoustic impedance of the material where the wave transmits to. Further note that the reflection coefficient can be negative if the wave propagates from a material of high impedance to low impedance, which accounts for possible negative values of the reflected pressure.

Using these definitions, the conservation of acoustic pressure (Equation 1) can be written in two ways, with each case depending on whether the transmitted wave (Equation 6) or reflected wave (Equation 7) may be used for further calculations.

$${P}_{i n c i d e n t} ( 1 + r ) = {P}_{t r a n s m i t t e d} \qquad ( EQ . 6 )$$

$${P}_{reflected} = {P}_{incident} ( t - 1 ) \qquad ( EQ . 7 )$$

As an acoustic wave travels through a medium, it loses pressure due to absorption and scattering. These losses are much higher in air than in a solid or liquid, so they must be accounted for when analyzing data from an air-coupled ultrasound experiment. This is mathematically represented as an exponential decay of the initial pressure when the acoustic wave was generated (Equation 8).

$$P ( x ) = {P}_{0} exp ( - α x ) \qquad ( EQ . 8 )$$

Here, α is the attenuation coefficient of the medium of acoustic wave propagation, x is the distance the wave traveled, and P₀ is the initial pressure of the wave.

### Model Derivation

The path of acoustic wave propagation in the experimental setup is shown in FIG. 1b. FIG. 1b is related to FIG. 1a, and is a representation of acoustic qualities of model derivation, with arrows indicating a general direction of wave propagation. Reflected waves are normal to the interface but shown at an angle for clarity.

For through-transmission, three interfaces are considered: 1) the air-film “front” interface, 2) the film-air “back” interface, and 3) the air-receiving transducer interface. Although the electrode-current collector interface may be considered, the individual responses of the electrode and current collector can be homogenized and considered as a single film, as the acoustic impedance of solids is much higher than that of air, thus showing negligible differences between the “front” and “back” interfaces.

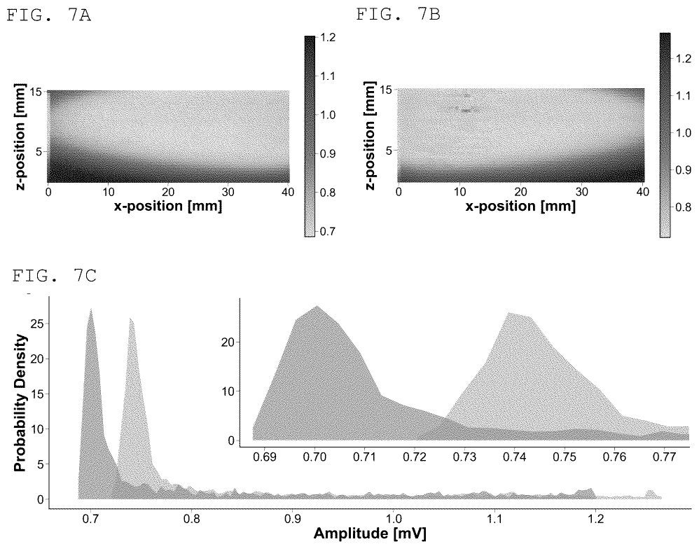

FIGS. 7A and 7B show experimental justification of these results. FIG. 7A shows an acoustic map of an electrode with the electrode facing the transmitter (“front” ultrasound wave goes through the electrode first). FIG. 7B shows an acoustic map of an electrode with the A1 current collector facing the transmitter (“back” ultrasound wave goes through A1 current collector first). FIG. 7C shows the corresponding amplitude distribution curves including a zoomed-in view.

For each interface, the conservation of acoustic pressure is applied (Equation 9) and the reflected pressure is represented in terms of the incident pressure (Equation 10) using Equation 6. Note that j is used as an index corresponding to each interface, and i indicates the incident pressure.

$${P}_{j , i} + {P}_{j , r} = {P}_{j , t} \qquad ( EQ . 9 )$$

$${P}_{j , i} ( 1 + {r}_{j} ) = {P}_{j , t} \qquad ( EQ . 10 )$$

For each path along a single medium, acoustic pressure loss is applied as Equation 11 by assuming that the incident pressure when a wave reaches an interface, P_{j,i}, is from the attenuation of the transmitted pressure from the previous interface, P_{j-1,t}. This relationship is key in representing the final pressure in terms of the initial pressure.

$${P}_{j , i} = {P}_{j - 1 , t} exp ( - {α}_{j} {x}_{j} ) \qquad ( EQ . 11 )$$

In this model, 3 interfaces were considered; thus, 3 sets of Equations 10 and 11 were used to define the observed pressure as follows: i) determine the incident pressure at interface with Equation 11 (P_{1,i}=P₀ exp(−α₁x₁) at the first interface), ii) apply Equation 10 (P_{1,t}=(1+r₁)P_{1,i}), iii) substitute P_{1,i} in the expression for P_{1,t} (P_{1,t}=(1+r₁)exp(−α₁x₁)P₀), and iv) calculate the incident pressure at the next interface by substituting Pit in Equation 11 for j=2 and repeat for every interface until the transmission of the signal into the transducer (j=3 in this case). Assuming the transmitted wave at the air-receiver interface, P_{3,b} is the observed pressure designated as P_{obs}, the equation for this quantity following substitutions using the transmitted pressure and pressure attenuation is given in Equation 12.

$${P}_{o b s} = ( 1 + {r}_{3} ) ( 1 + {r}_{2} ) ( 1 + {r}_{1} ) exp ( - {α}_{1} {x}_{1} ) exp ( - {α}_{2} {x}_{2} ) exp ( - {α}_{3} {x}_{3} ) {P}_{0} \qquad ( EQ . 12 )$$

A simpler expression can be determined for an unobstructed path of the acoustic wave through the medium as given in Equation 13.

$${P}_{a i r} = ( 1 + {r}_{3} ) exp ( - {α}_{a i r} {x}_{1} ) exp ( - {α}_{a i r} {x}_{2} ) exp ( - {α}_{a i r} {x}_{3} ) {P}_{0} \qquad ( EQ . 13 )$$

Taking the ratio of the amplitudes for the through-film and through-air pressures simplify the expression to Equation 14 given α₁ and α₃ are the same as αₐᵢᵣ.

$${P}_{o b s} / {P}_{a i r} = ( 1 + {r}_{2} ) ( 1 + {r}_{1} ) exp ( ( {α}_{a i r} - {α}_{2} ) {x}_{2} ) \qquad ( EQ . 14 )$$

The reflection coefficients are defined in Equations 15 and 16 where r₂ can be substituted with −r₁ as justified by film homogenization and the definition of the reflection coefficient. This can be justified by expressing the reflection coefficients in terms of the acoustic impedance of sample materials. For example, given an acoustic impedance of air to be 415 Rayl (kg/m²/s) and of aluminium to be 17 MRayl, the r₂ value is −0.99995. The acoustic impedance of porous ceramic materials, ranging from 3 to 40 MRayl, may approximate that of a battery porous electrode. In all cases, the reflection coefficients of the solids are significantly larger than air.

$${r}_{1} = ( {Z}_{film} - {Z}_{a i r} ) / {Z}_{f i l m} + {Z}_{a i r} ) \qquad ( EQ . 15 )$$

$${r}_{2} = ( {Z}_{a i r} - {Z}_{f i l m} ) / {Z}_{a i r} + {Z}_{f i l m} ) = - {r}_{1} \qquad ( EQ . 16 )$$

The reflection coefficient, r₁, can then be calculated from P_{obs} using Equation 17 and subsequently used to determine the acoustic impedance of the film with Equation 18.

$${r}_{1} = ( 1 - {P}_{o b s} / {P}_{a i r} exp ( {α}_{f i l m} - {α}_{a i r} ) {x}_{film} ) \hat{} 1 / 2 = {r}_{f i l m} \qquad ( EQ . 17 )$$

$${Z}_{film} = {Z}_{a i r} ( ( 1 + {r}_{f i l m} ) / ( 1 - {r}_{f i l m} ) ) \qquad ( EQ . 18 )$$

This quantity becomes the basis for analyzing the results presented on film density (Equation 19) as the speed of sound through the film, c_{film}, is asserted to be constant. Similar equations have been derived to calculate the acoustic impedance; however, air is a strongly attenuating medium, so its impact on attenuation losses is emphasized here.

$${p}_{f i l m} = {Z}_{f i l m} / {c}_{f i l m} \qquad ( EQ . 19 )$$

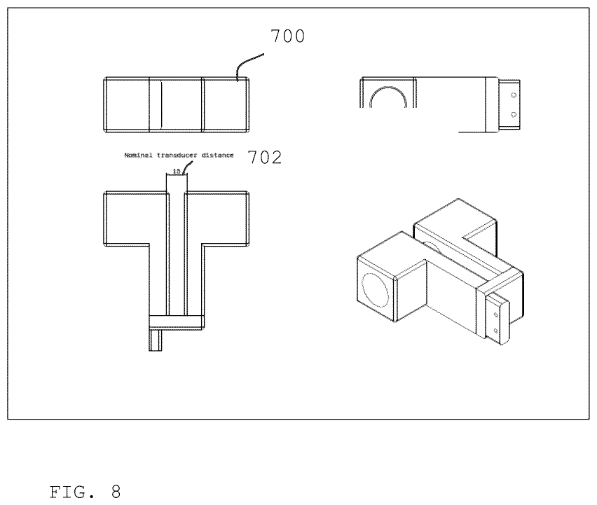

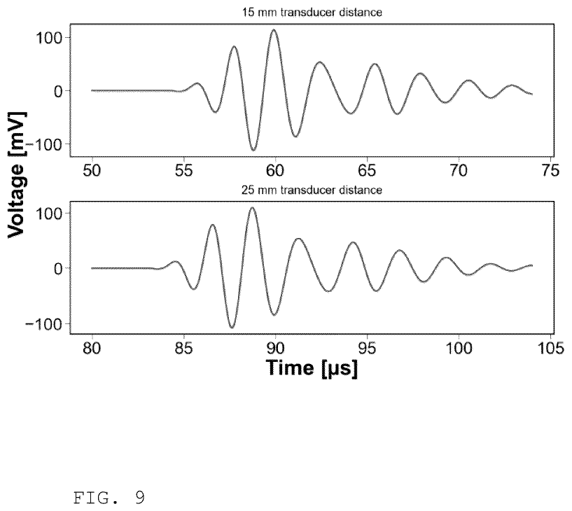

Experimental values and calculations are provided in FIGS. 7-8. FIG. 8 shows drawings of a transducer holder 700 with a minimal transducer distance 702, preferably of 15 mm. FIG. 9 shows an ultrasound signal from transmission through air with transducer distances of 15 mm (top) vs 25 mm (bottom). Regarding the determination of speed of sound (or time-of-flight, ToF), Biot theory predicts that for porous materials, the observed speed of sound can tend towards the speed of sound in the filling media. In this case, the sound speed in the porous electrode would be near that of the speed of sound in water, which was experimentally observed. Due to the weak amplitude observed when the wave from the air-coupled transducer passes through the film, the level of noise affects the calculation of the wave arrival time used for the ToF. Thus, the ToF through the electrode cannot be accurately or precisely measured. Instead, since it has been observed that no significant shift happens as the wave passes through the electrode, then the speed of sound of the electrode immersed in an air environment may not significantly differ to the speed of sound in air.

### Results and Discussion

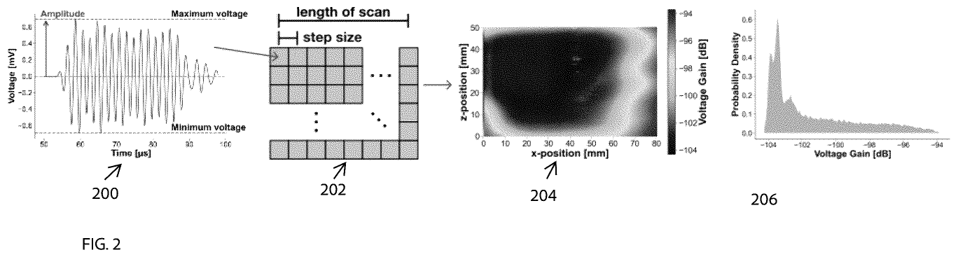

Ultrasound waves transmitted through electrode films were recorded at every point of a grid set by steps in the x and z-directions of a 3D printer gantry. A schematic of this process is shown in FIG. 2. FIG. 2 shows an example ultrasound waveform 200 collected at one position of a raster scan, demonstrated by the grid 202, and the resulting acoustic voltage gain map 204, and voltage gain distribution (probability density) 206 after post-processing.

The amplitude of each waveform, P_{obs}, is defined as half the difference between the maximum and minimum voltages of the wave packet. The gain was then taken relative to the amplitude of the unobstructed wave propagated through air, Pair, and expressed in decibels (dB) via EQ. 20.

$$Voltage gain = 20 {log ( {P}_{o b s} / {P}_{a i r} )}_{V} \qquad ( EQ . 20 )$$

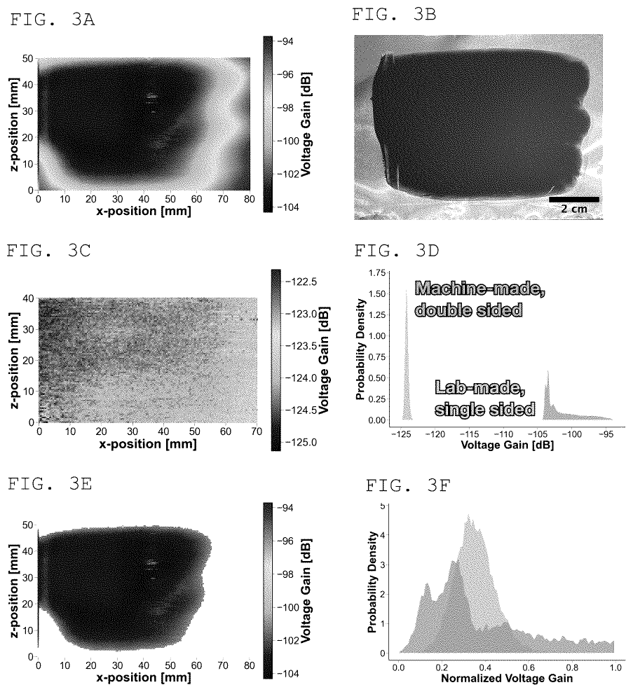

FIGS. 3A-3C demonstrate the viability of using the voltage gain as the mapped metric by comparison with an optical image. FIG. 3A shows a map of voltage gain for a lab-cast electrode. FIG. 3B shows an optical image of the lab-cast electrode. FIG. 3C shows an acoustic image of a double-sided, machine-made NMC electrode. FIG. 3D shows the voltage gain distribution comparison between the machine-made and the lab-made electrode, showing a clear difference in voltage gain distribution. FIG. 3E shows the most homogeneous region of the lab-made electrode from FIG. 3A. FIG. 3F shows the voltage gain distribution of the most homogeneous region of the lab-made electrode versus the machine-made electrode, where the lab-made electrode continues to show a more skewed distribution.

While the optical image may have better spatial resolution, it may not capture local variations in thickness or mass loading over a specified area, as electrodes are generally opaque. A substantial area with voltage gain between −104 dB to −100 dB appears in the acoustic image with a similar shape as that of the electrode, possibly indicating a portion of the acoustic map that visually corresponds to the electrode. A lower voltage gain reflects a lower amplitude, potentially caused by enhanced acoustic scattering from densely packed particles, implying higher local mass loading. The gradients observed at the voltage gain transitions from about −95 dB to −100 dB around the electrode film edge are attributed to an artifact of the scanning process due to the relatively large size of the acoustic transducers compared to the sharp features at the film edge. The gradient may also be affected by the transition from a lower mass loading to a higher mass loading. Subsequent analyses of the value distributions will account for this error, but appreciable differences can still be observed.

A machine-made electrode is shown in FIG. 3C. Note that the voltage gain of the machine-made electrode is lower than that of the lab-cast ones, possibly due to higher density of active material (2.0-3.0 g/cm³ tap density for lithium nickel manganese cobalt oxide (NMC) 22 compared to 0.7 g/cm³ tap density for lithium manganese iron phosphate (LMFP) 23) or densification from calendering. By comparing the voltage gain distribution in FIG. 3D, the machine-made electrode shows a sharp peak whereas the lab-made electrode shows a peak and a tail. The tail could be indicative of the averaging effect as the transducer covers the electrode and current collector regions, and the apparent peak would indicate the region of only the electrode consistent with the electrode-only surface of the machine-made electrode (no current collector regions in the scan area). The machine-made electrode appears to be uniform based on the narrow voltage gain distribution. On the other hand, the lab-made electrode does not appear to be uniform as shown in the skewed distribution. Even when the relevant portion of the electrode is isolated as shown in FIG. 3E, the distribution of voltage gain is still observed to be skewed. This observation is made explicit in FIG. 3F where the values of the isolated electrode region appears to be skewed towards lower voltage gain values following a tail as opposed to an almost normal distribution with the machine-made electrode. These results suggest that acoustic mapping enables the identification of homogeneous regions and detection of mass loading irregularities. With further refinement, this approach could guide electrode slitting to minimize low loading areas and maximize the area of desired mass loading.

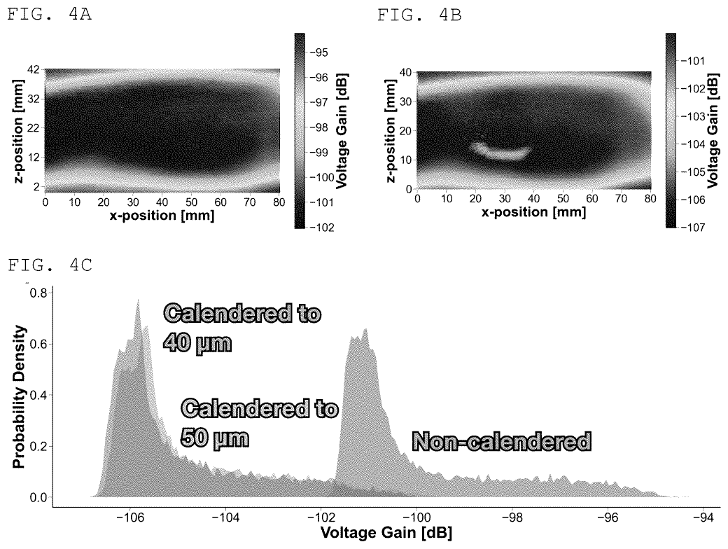

Calendering can also be detected since it is a physical modification of the electrode density. This process involves passing the electrode through rollers with a set gap to compress and compact the electrode to a specific thickness and consequently porosity. FIGS. 4A and 4B show the same electrode (4A) as-cast and (4B) after calendering using a 40 μm gap. Calendering clearly shifted the voltage gain values of the electrodes (FIG. 4C). Calendering at 40 μm induced an anomalous “island” in the lower-right quadrant of FIG. 4B, possibly because of wrinkling of the electrode from uneven rollers or the electrode snagging on the calendering machine. The voltage gain distribution shifted to lower values after calendering to any thickness. The initial loose packing of the electrode allowed unconstrained vibrations into the void space. As the electrode is calendered, the void space is replaced by stiffer solid materials and hindered vibrations as manifested by a lower amplitude. This implies that the electrode was densified as the compacted particles impeded the vibration of the acoustic wave. The smaller shift by increasing the calendering gap by 10 μm suggests that the electrode was almost fully compacted after one pass at a 50 μm gap, and any subsequent calendering may only marginally densify the electrode and can potentially induce defects.

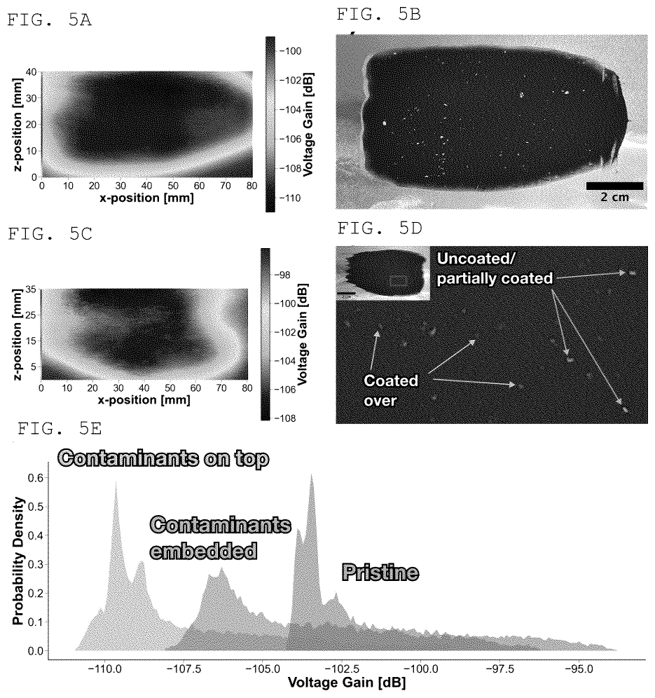

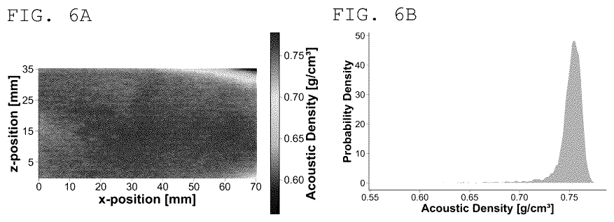

Ultrasound can also be applied for defect detection in electrodes, particularly for metal contaminants. Metal contaminants have been demonstrated to act as sites for internal shorting and significantly reduce long-term cyclability. Furthermore, internal shorting is linked to thermal runaway, which can lead to combustion and other safety hazards. Thus, detection of contaminants may be used in the electrode and cell fabrication processes. FIGS. 5A-5D shows the acoustic images and associated optical images of aluminum shavings on top of the electrode (FIGS. 5A, 5B) and embedded shavings (FIGS. 5C, 5d). When metal contaminants are on top of the electrode, optical images provide better detection due to contrast and resolution. On the other hand, the acoustic image cannot resolve the small metal contaminants, which is likely due to feature broadening as discussed previously.

However, it is observed that the range of voltage gain changes when contaminants are present in the electrode as shown in FIG. 5E. This implies a decrease in the observed amplitude through the film which physically manifests as a stiffness mismatch between the materials along the interface; the stiffer metal specks dampen the initial vibration from the air more than the electrode composite, thus propagating a wave with a lower amplitude. This can be encapsulated as the reflection or transmission coefficients of the interface, which indicate that this interface is more reflective than transmissive. Protrusions from the metal contaminants may cause the acoustic wave to impinge the film at an angle (oblique incidence) and disperse the energy in other directions and propagation modes, thus further decreasing the observed amplitude. This shift of voltage gain range within the electrode can then be used to indicate the presence of contaminants for quality control by comparing to a standard gain range from an electrode with no contaminants.

A similar analysis can be done when the contaminants are embedded in the electrode. Note that some contaminants are still visible, as it is difficult to contain all the particles at the bottom of the electrode. Metal specks that were coated over can barely be seen in the optical image, but the acoustic image provides an immediate indication that contaminants are present. This is clearly shown by the probability density distribution of the voltage gain shifting to lower values. Although the film has metal particles embedded, it may be treated as having an air-electrode interface. The metal specks would then likely act as points of concentrated mass, which increase the acoustic density, as speculated for FIG. 3A. However, the incremental acoustic density is not as much as having the contaminants on top of the electrode, possibly due to having a rough solid-solid interface (electrode-current collector) instead of a rough solid-air interface, which could scatter better. Given that the resulting acoustic density range extends beyond that of uncontaminated films, the acoustic mapping strongly suggests that contaminants are present in the electrode.

Observations from the calendering experiment suggest a link between the acoustic response of the material and its density. A simple model may be used to derive the acoustic density of the electrode film from the signal amplitude. These values were compared to the geometric density of the electrode, taken by punching out disks from the electrode sheet and measuring their mass and thickness. A density range was generated by measuring at different calendering levels was shown to have observable changes. The average geometric density of the electrode was found to be about twice the average acoustic density, consistent with Biot theory, which suggests that the observable density of a porous solid lies between that of the pure solid and that of the medium.

Non-contact ultrasound was shown to be a promising NDE technique for battery electrodes to reduce product waste, improve quality control, and increase confidence in battery safety. The basic model of ultrasound wave propagation through air and an electrode film enables both quantitative and qualitative analysis of electrodes, highlighting different electrode treatments and comparisons. Ultrasonic scans show mass distributions and inhomogeneity throughout the electrode, an advantage over optical images, regardless of whether it is lab-fabricated or machine-made. Calendering can be observed as a decrease in the voltage gain of the electrode. Metal contaminants also decrease the voltage gain of the electrode, both when visibly on top and embedded within, an advantage over existing optical methods. The output of a simple acoustic density model predicts values close to the expected density.

### Experimental Results

### Electrode Fabrication

Electrodes were fabricated with lithium manganese iron phosphate (LMFP, MSE Supplies), polyvinylidene fluoride (PVDF, MSE Supplies) dispersed in N-methylpyrrolidone (NMP, Thermo Scientific) at 10% by weight and carbon black (Ketjenblack EC600JD, MSE Supplies) at a 90:5:5 weight ratio respectively, in 500 mg batches. Components were dispersed in NMP and mixed using a centrifugal mixer (THINKY USA) at 2000 rpm for 15 minutes. A doctor blade was used to cast the resulting slurry onto an Al current collector (MSE Supplies) using a film coater (TOB-VFC-150). Electrodes were dried at 80° C. in a vacuum oven (TOB-6050) for at least one hour or until solvent has evaporated. Electrodes were calendered (TOB-SG-100L) as necessary with a nominal gap of 50 μm. Metal contaminants were emulated by filing Al foil and spreading on top of the electrode right after casting or spread onto the current collector right before casting. NMC electrodes were purchased from MTI Corporation.

### Ultrasound Scanning

Air-coupled transducers (NCG500-D13 as receiver, NCG500-D19 as transmitter, The Ultran Group) were used to perform ultrasound scans with a custom setup, using a tone-burst pulser (USB-UT350, Ultratek) to provide more energy per wave. Individual electrodes were placed in a 3D-printed film holder attached to a modified 3D printer plate (Creality Ender-3 3D Printer). A scanning area was determined by the area of interest on the electrode, the diameter of the transducers, the transducer holder, the dimensions of the electrode, and the film holder such that the entire scanned area covered by active portion of the transducer faces would have minimal interaction with the film holder or areas without the electrode film, and to avoid crashing the transducers onto the plate. The scanned areas were no more than 100 mm in the x-direction and 50 mm in the z-direction, with each direction scanned with a step size of 0.5 mm. The transmission ultrasound signal at every point was the average of 1000 sample waves comprising 1000 points recorded over a 48.000 us period with a delay of 49.000 us upon the initial wave pulse. The peak frequency of the transducers was 0.5 MHz. The tone-burst pulser was set to 32 half-cycles.

### Justification for Electrode Film Homogenization

The reflection coefficients of the electrode film from the front and back are assumed to be equal and opposite (r₂ can be substituted for −r₁), as in EQ. 15 and EQ. 16. To support this claim, the reflection coefficients can be expressed in terms of the acoustic impedance of the relevant materials (e.g. electrode and air, or air and aluminum). The acoustic impedance of air and aluminum were taken to be 415 Rayl (kg·m⁻²·s⁻¹) and 17 MRayl, respectively, yielding an r₂ value of −0.99995. The acoustic impedance of the electrode itself was approximated using values for porous ceramic materials which ranged from 3 to 40 MRayl, leading to r₁ of close to 1. Since the reflection coefficients of the solids (aluminum, electrode estimate) are 1) significantly larger than air and 2) are of the same magnitude, it is expected that the reflection coefficients have a values near 1 or −1, validating EQ. 15 and EQ. 16. Further, scans of the same electrode were taken, one with the electrode film facing the transmitter (“front”) and the other with the current collector facing the transmitter (“back”) shown in FIGS. 7A and 7B, respectively. The relative error between the two scans did not differ by more than 6% regardless of statistical metric or chosen scan as reference. Regardless, the difference in acoustic density measured from both configurations is effectively negligible, allowing similar conclusions to be drawn from either. For all experiments in the main text, the electrode was in the “front” configuration.

### Calculation of Ultrasound Parameters

The speed of sound is approximated by using the collection step of the experiments (every 48 ns) as the ToF shift. The collection frequency could be increased to every 2 ns, but the signal-to-noise ratio worsens and obstructs clear determination of the approximate ToF. By this logic, the speed of sound in the film can then be calculated by assuming that the path length that acoustic waves travel before getting recorded is constant (EQS. 21-23). Given the speed of sound through air is 343 m/s and assuming Δt to be at most 50 ns and the range of x2 to be 30 to 50 μm, the approximated sound speed through the film is between 522.07 to 800.79 m/s. A smaller time shift approximates the sound speed to be closer to that of air while a larger shift tends to faster sound speeds. For all calculations, the speed of sound through the film was taken as 522.07 m/s as a “best case” scenario. The speed of sound through the film can also be approximated as the ratio of the acoustic impedance to the apparent density. The apparent density is measured by sampling the and the acoustic impedance can be calculated using ultrasound data. The average acoustic impedance was measured to be about 412000 kg·m⁻²·s⁻¹ corresponding to an apparent density of about 1.33 g/cm³ which would correspond to a speed of sound of about 310 m/s. This value is at least half of the time-domain approximated speed of sound through the film, thus necessitating more precise methods. Regardless, the values from the two methods are of the same magnitude and can be used to approximate the density of the film.

$$Δ t = {t}_{air only} - {t}_{with electrode} \qquad ( EQ . 21 )$$

$$Δ t = ( x 1 + x 2 + x 3 ) / {c}_{a i r} - ( x 1 / {c}_{a i r} + x 2 / {c}_{f i l m} + x 3 / {c}_{a i r} ) \qquad ( EQ . 22 )$$

$${c}_{f i l m} = ( 1 / {c}_{a i r} - Δ t / {x}_{2} ) \hat{} - 1 \qquad ( EQ . 23 )$$

### Technique

The capability described herein uses an air-coupled ultrasonic scanning platform for non-contact metrology of battery electrodes. Rather than focusing on SOC or SOH within an assembled battery undergoing cycling and aging, the focus here is on developing a non-contact, data-rich metrology tool that can map local variations in density and thickness and detect defects during electrode casting and drying. Piezocomposite air-coupled transducers (Ultran) operating at 0.5 to 1 MHz provide sufficient air coupling to enable sound waves to travel through air from the transmitting transducer, through the thin-film electrode coated on a metal current collector, and then into the receiving transducer. The platform includes an X-Y gantry for rastering the transducers at a best resolution of 0.1 mm, a holder for the thin film electrode, and a holder for the ultrasonic transducers. The transmitting transducer is connected to an ultrasonic tone burst pulser (Ultratek), which generates a high-energy ultrasound wave. The receiving transducer is connected to a portable oscilloscope (Picoscope 2208B) with sampling rate at 1 Gigasample per second. The inventors have developed Python code to control the pulser and oscilloscope, including a graphical user interface (GUI), and data analysis functions.

Further signal processing algorithms are being developed to better understand and differentiate between different types of defects (buried voids, agglomerates and clumps, thickness, metal contaminants) based on wave shape analysis in both time- and frequency-domain. The platform is designed to be modular and can scan films of different sizes and shapes, and serves as a proof-of-concept for what could be a rapid, in-line metrology tool placed within a roll-to-roll electrode fabrication line.

While the invention has been described with reference to the embodiments above, a person of ordinary skill in the art would understand that various changes or modifications may be made thereto without departing from the scope of the claims.

1. An air-coupled ultrasonic metrology platform for non-destructive, in-line characterization of battery electrodes, comprising:
a transmitting piezocomposite air-coupled ultrasonic transducer;
a receiving piezocomposite air-coupled ultrasonic transducer;
a holder for securing a thin film electrode coated on a metal current collector in an acoustic path between the transmitting and receiving transducers;
an X-Y raster scanning gantry configured to move the transducers relative to the electrode;
a tone-burst pulser coupled to the transmitting transducer for generating a high energy ultrasound signal;
a digital oscilloscope coupled to the receiving transducer for capturing the transmitted ultrasound signals; and
a processor configured to execute a signal processing algorithm that generates voltage gain maps of the electrode based on the received signal amplitude, defined as half of the difference between the maximum and minimum voltage of the wave packet, relative to the amplitude of the unobstructed wave propagated through air or a calculation of acoustic density based on an estimated sound speed.

2. The platform of claim 1, wherein the transmitting and receiving transducers are spaced at a fixed distance of 15 mm.

3. The platform of claim 1, wherein the signal processing algorithm determines acoustic density using the reflection coefficient calculated from exponential attenuation in air and the impedance mismatch between air and the electrode film.

4. The platform of claim 1, wherein the voltage gain or acoustic density map reveals local variations in mass loading, porosity, or thickness of the electrode film.

5. The platform of claim 1, wherein the signal processing algorithm includes time-domain and frequency-domain waveform analysis to detect at least one of: buried voids, metal contaminants, agglomerates, or density gradients.

6. The platform of claim 1, wherein the transducers are mounted using a 3D-printed transducer holder with minimum spacing and alignment constraints to avoid interaction with the electrode holder.

7. The platform of claim 1, wherein the oscilloscope has a sampling rate of at least 1 gigasample per second.

8. The platform of claim 1, wherein the transducers are raster scanned over an area up to 100 mm×50 mm with a step size of 0.5 mm or smaller.

9. A method of non-destructively characterizing a battery electrode using an air-coupled ultrasonic scanning system, the method comprising:
(a) transmitting an ultrasonic wave through air from a transmitting transducer to a receiving transducer, the wave passing through a thin film battery electrode coated on a metal current collector positioned between the transducers;
(b) acquiring a signal waveform at the receiving transducer;
(c) determining an observed pressure based on a peak-to-peak amplitude of the waveform;
(d) determining the voltage gain from the observed pressure;
(d) calculating an acoustic impedance of the electrode based on the observed pressure and a modeled air attenuation path; and
(e) determining an acoustic density of the electrode by dividing the acoustic impedance by a predetermined speed of sound through the film.

10. The method of claim 9, further comprising creating a raster scan of the electrode surface and generating a two-dimensional voltage gain map.

11. The method of claim 9, wherein the acoustic impedance is calculated by modeling the observed pressure as a function of exponential air attenuation and impedance mismatch at air-film interfaces, in order to arrive at an acoustic density value.

12. The method of claim 9, wherein the calculated voltage gain is used to compare the homogeneity of lab-cast and machine-cast electrodes based on probability density distributions.

13. The method of claim 9, wherein the presence of metal contaminants in the electrode is identified based on a decrease in the range of voltage gain values.

14. The method of claim 9, wherein changes in voltage gain after electrode calendering are used to infer densification and porosity variations across the electrode surface.
